(19) United States
(12) Patent Application Publication
(10) Pub. No.: US 2002/0052300 A1
(43) Pub. Date: May 2, 2002

(54) PROCESS FOR PREPARING SOAP MATERIAL

(76) Inventors: HERMANUS CHRISTOFFEL KEMPER, BRIELLE (NL); JACOB OOSTERMAN, SPIJKENISSE (NL)

Correspondence Address: PATENT & TRADEMARK ADMINISTRATOR, ICI AMERICAS INC LAW DEPARTMENT, PO BOX 8340, WILMINGTON, DE, 19803-8340, US

(21) Appl. No.: 09/242,426

(22) PCT Filed: Jul. 23, 1997
(86) PCT No.: PCT/EP97/04017

(30) Foreign Application Priority Data
Aug. 16, 1996 (EP) 96202296.8

(51) Int. Cl. A61K 7/50; A61K 7/075

(52) U.S. Cl. 510/141; 510/152; 510/458

(57) ABSTRACT

A method is provided for enabling the use of high levels of fluid material (e.g. emollients and moisturizers) in the preparation of soap material (e.g. bars and noodles), whilst the soap material has a firm, smooth and homogeneous appearance. In testing, the resulting soap bars appeared to be exceptionally mild to the skin. The method comprises the use of a granular material comprising a fluid phase and a particulate material. In an alternative manner, the fluid phase and particulate material may be incorporated into the mixture separately.

The present invention relates to a process for preparing soap material, in particular bars, in which a high level of fluid materials is included. Also, the invention relates to a granular product comprising a particulate carrier material and a functional soap ingredient which is a liquid or pasty material under ambient and/or processing conditions, for application into a solid soap bar.

In general, soap bars (either for personal wash or other purposes) contain a number of functional ingredients, such as, for example surface active agents, wetting agents, moisturizing agents, perfumes, colours, etcetera. Some of these ingredients are solid, others are fluid (liquids) and still others are in the form of a pasty material.

In the preparation of a soap bar, the final stage is generally pressing the complete mixture of all ingredients into a shape. Prior to this, a mixing operation is needed in order to have all the desired ingredients into the final mixture before pressing it into shape. In general, mixing is effected by subjecting the mixture of ingredients to shear, by using mixing apparatus like double screw extruders, cavity transfer mixers and the like. It is a known problem in the field of mixing soap ingredients, that mixing can be insufficient it not enough shear can be imparted to the mixture of ingredients, resulting in soap bars of unsatisfactory quality and appearance. This is especially the case, if one or more soap ingredients are in a liquid or pasty form. It is thought that such liquid or pasty ingredients act as a lubricant, and thus reduce the amount of shear imparted to the mixture. The above problem is also occurring when the producer of soap bars is compounding himself soap noodles (which contain raw soap ingredients, such as fatty acid salts, free fatty acids, free alkali, polyols, salts, glycerol), with one or more fluid or pasty ingredients like water, glycerol, liquid free fatty acids, liquid fatty acid esters, liquid surfactants, dissolved surfactants, mineral oils, silicon oils, emollients, moisturizers, humectants, perfumes or mixtures thereof. In that case, it is a general problem that not enough shear can be imparted to the mixture of noodles and additional (fluid/pasty) ingredients, resulting in poor mixing and subsequent soap bars of poor quality.

Although it is in some cases possible to reduce or limit the application of liquid or pasty ingredients when manufacturing a soap bar, it is not always desired or possible to replace fluid or pasty ingredients for solid ones. Additionally, there is an increasing demand for soap bars containing more water, glycerol, liquid free fatty acids, liquid fatty acid esters, liquid surfactants, dissolved surfactants, mineral oils, silicon oils, emollients, moisturizers, humectants, perfumes or mixtures thereof. Unfortunately, such compounds are often fluids, and hence, the problem of obtaining a homogenous mixture in mixing soap ingredients is even bigger.

Thus, there is a demand for a process for the preparation of soap bars enabling the use of a relatively large proportion of liquid or pasty ingredients, whilst good mixing is possible to result in a homogenous mixture. Furthermore, there is a demand for a method which enables mixing (raw) soap noodles with one or more fluid or pasty ingredients, still yielding soap bars of a good, homogeneous quality.

Surprisingly, it has now been found that the above can be achieved by using a process for the preparation of a soap material, which process comprises the steps of mixing:

a solid phase and

a granular material, characterized in that the granular material comprises at least one fluid phase and a particulate material.

In an alternative way, the above mentioned objectives can also be achieved by a process for the preparation of a soap material comprising the steps of mixing:

a solid phase,

a fluid phase, and characterized in that

a particulate material is incorporated into the mixture.

In comparison to the above mentioned process, the separate preparation of granules (comprising a fluid and a particulate carrier material) is avoided. However, the results so obtained can be very satisfactory as well.

Although the inclusion of a particulate material in soap bars is known, it has always been used for a different purpose, and the way it is employed is also different. For example, in WO 96/06595, cleaning compositions (mainly liquid, but also in bar form) are disclosed which contain a particulate material of a specified size, of which the purpose is to increase the cleansing effect, by way of abrasion. In JP 61/195200, cast soap bars (also called framed soap) are disclosed which exhibit a moisturizing feeling, without the foaming being affected by the presence of superfatting agents.

Using the process according to the invention, the amount of fluid so introduced into the soap material is preferably at least 10% by weight, based on the total weight of soap material so produced. More preferably, the total amount of fluid is at least 20% by weight, based on the total weight of soap material so produced. Even more preferred the total amount of fluid is at least 25% by weight, based on the total weight of soap material so produced. Most preferred the total amount of fluid is at least 35% by weight, based on the total weight of soap material so produced.

Therefore, the invention embraces the use of a particulate carrier material in combination with the use of high levels of fluid in the preparation of a solid soap material. The preferred lower limits for the fluid level in this respect are set out in the paragraph above.

Preferably, the total amount of fluid is at most 60% by weight, based on the total weight of soap material so produced. More preferably, the total amount of fluid is at most 50% by weight, based on the total weight of soap material so produced. Most preferred such a maximum level of fluid is at most 40% by weight, based on the total weight of soap material so produced.

The process according to the present invention can be used to incorporate a wide range of different liquid or fluid (or even pasty) materials in the soap material, but the method is particularly useful for incorporating fluids like water, glycerol, liquid free fatty acids, liquid fatty acid esters, liquid surfactants, dissolved surfactants, mineral oils, silicon oils, emollients, moisturizers, humectants, perfumes or mixtures thereof. All such compounds are known in the art of manufacturing cleansing bars for personal care. The method according to the present invention is especially useful for incorporating moisturizers and emollients which comprise fatty acid esters.

For the purpose of the invention, with a liquid or fluid ingredient are herein to be understood to be compounds which are liquid or fluid at the conditions (temperature and pressure) of processing (i.e. mixing) and/or at room temperature and pressure.

The amount of granules and/or particulate material to be used in the process according to the invention depends e.g. on the amount and type of the fluid to be incorporated, the desired appearance of the final soap material, the manner of processing, etcetera. However, in general the amount of granules and/or particles will be chosen such that the total amount of particulate material in the soap material so produced is at least 2% by weight, based on the total weight of soap material so produced. It is more preferred that the total amount of particulate material in the soap material so produced is at least 5% by weight, even more preferred at least 10% by weight, based on the total weight of soap material so produced. For some purposes, it can be desired that the total amount of particulate material in the soap material so produced is more than 20% by weight, based on the total weight of soap material so produced.

Depending e.g. upon the composition of the granules and the desired level of fluids in the final soap material, the amount of granules to be included into the soap material will be from 0.5 to 60% by weight, based on the final soap material. More preferably, said amount will be from 2-50% by weight, and most preferred from 5-40% by weight.

The nature of the particulate material can vary to a large extend. It was found, however, that for the purpose of the invention it is preferred that the particulate carrier material comprises at least a part or total of an anorganic material. Preferred anorganic materials in this respect are talc, finely ground natural calcium carbonate from limestone, precipitated calcium carbonate, clay, titanium dioxide, silicas/silicates, alumina or combinations of the above.

Regarding the solid phase or fraction, this the part of the soap material may typically contain (in part or all of) the surface active agents or emulsifiers, including anionic-, cationic-, nonionic-, amphoteric, zwitterionic-detergents, fatty acid salts, synthetic detergents etcetera.

Although the method can be used for translucent or transparent soap bars, it is particularly suitable in the manufacture of opaque soap material.

The present invention can be used for the preparation of soap material in any form or shape. Generally, such soap material is a soap bar, noodle, pellet, flake, or powder.

Part of the present invention are the granules which can be used in the process as set out above. Such granules comprise a fluid phase and a particulate material, wherein the fluid phase preferably constitutes more than 20% by weight of the granules, more preferably more than 40% by weight of the granules. Of such granules the fluid phase preferably comprises water, glycerol, liquid free fatty acids, liquid fatty acid esters, liquid surfactants, dissolved surfactants, mineral oils, silicon oils, emollients, moisturizers, humectants, perfumes or mixture thereof. Regarding emollients and moisturizers, fatty acid esters are preferred.

Of said granules, the particulate carrier material preferably comprises an anorganic material. Preferably, said anorganic material comprises talc, finely ground natural calcium carbonate from limestone, precipitated calcium carbonate, clay, titanium dioxide, silicas/silicates, alumina or combinations of the above.

The invention is now exemplified by the following examples, which are not to be seen as limiting the scope of the invention.

### EXAMPLE 1

Granules were prepared according to the following method, using a range of different carrier materials (see table below). The required amount of particulate carrier material (40.0 gram) was dosed into a beaker and dispersed for 2 minutes using a spoon. Following this, the required amount of fluid (25.0 gram in total) was added. The fluid added was a mixture of 9 gram of PRISORINE GTIS 2041 (glyceryl tri-isostearate, as marketed by Unichema International) and 16 gram of ESTOL E03 GC 3606 (PEG 7 glyceryl cocoate, as marketed by Unichema International). Mixing was continued for another 5 minutes, using a spoon.

The following particulate carrier materials have been tested:

| | | |
|---|---|---|
| 1 | Neosyl GP | amorphous silicium dioxide, particle |
| | | diameter 18 micron, as marketed by |
| | | Crosfield, United Kingdom |
| 2 | Neosyl TS | amorphous silicium dioxide, particle |
| | | diameter 12 micron, as marketed by |
| | | Crosfield, United Kingdom |
| 3 | Alusil ET | sodium aluminium silicate (zeolite), as |
| | | marketed by Crosfield, United Kingdom |
| 4 | Aerosil 200 | sodium aluminium silicate (zeolite), as |
| | | marketed by Degussa, Germany |
| 5 | Flowlac | spray-dried alpha-lactose, monohydrate, |
| | | as marketed by Meggle GmbH, Germany |
| 6 | Granulac 200 | granulated alpha-lactose, monohydrate, |
| | | as marketed by Meggle GmbH, Germany |
| 7 | Talcum PE 8454 | talcum powder, as marketed by Luzenac |
| 8 | Kaoline | hydrated aluminium silicate, Al₂O₃. |
| | | 2SiO₂.2H₂O as marketed by Acros |
| 9 | magnesium silicate, | as marketed by Molen Chemie, the |
| | | Netherlands |

The granules so obtained had a dry, free flowing and even appearance, and contained approximately 38% by weight of fluid).

For testing the efficacy of these granules in delivering fluids in basic soap mixtures, the granules were mixed with base soap noodles (PRISAVON 9226, Unichema International) having the following composition:

| | |
|---|---|
| sodium palmate | 49% |
| sodium palm kernelate | 34% |
| water | to the balance (about 10%) |
| palm/palm kernelate fatty acids | 6.5% |
| sodium chloride | 0.5% |
| glycerine | 0.2% |
| tetra sodium EDTA | 0.02% |
| tetra sodium etidronate | 0.02% |

The above soap noodles (3500 gram) were mixed with the granules (1500 gram) using an amalgamator (5 minutes), after which the resulting mixture was extruded, and pressed into final soap tablets. All resulting tablets had a firm, smooth and homogeneous appearance. In testing, the resulting soap bars appeared to be exceptionally mild to the skin, due to the large amounts of specific moisturizing fluids present in the final bars.

### EXAMPLE 2

Three of the carrier materials as listed above (Neosyl GP, Neosyl TS, and Alusil N) have been tested in the preparation of granules containing a higher level of fluid than in example 1. In Example 2, the same method of preparation was followed, apart from the following modifications:

60 gram of fluid mixture contained 40 gram of ESTOL E03 GC 3606 and 20 gram of PRISORINE GTIS 2041

in the preparation of the granules, 40 gram of particulate carrier material was mixed with 60 gram of fluid. (Hence, the granules so prepared contained 60% by weight of fluid).

The granules had a similar appearance as in example 1. The granules were used in the preparation of soap bars in the same manner as in example 1. Again, the resulting tablets had a firm, smooth and homogeneous appearance. In testing, the resulting soap bars appeared to be exceptionally mild to the skin, due to the large amounts of specific fluids present in the final bars.

### EXAMPLE 3

Two of the carrier materials (Neosyl GP/ Talcum PE 8454) as listed above and one fluid were added directly to soap noodles. Amounts of materials used were:

3885 g PRISAVON 9226 (Unichema International, composition see above),

280 g of Talcum PE 8454 (Luzenac)

335 g of Neosyl GP (Crosfield).

The above ingredients were dosed in a amalgamator, whereafter 500 g of ESTOL E03 GC 3606 (PEG 7 glyceryl cocoate, Unichema International) was stepwise added in 5 minutes under continuous mixing. After all fluid was added, mixing was continued for another 2 minutes. The mixture was extruded in a duplex refiner/plodder to prepare soap tablets.

All resulting tablets had a firm, smooth and homogeneous appearance. In testing, the resulting soap bars appeared to be exceptionally mild to the skin, due to the large amounts of specific moisturizing fluids present in the final bars.

### EXAMPLE 4

To prepare a soap material with an increased level of fluid, the following compounds were mixed in an amalgamator for 1 minute:

4000 g of PRISAVON 9240 (Unichema International), in flake form (prepared by using a roll-mill)

250 g Talcum PE 8454 (Luzenac, see above).

The soap flakes PRISAVON 9240 had the following composition:

| | |
|---|---|
| sodium palmate | 60% |
| sodium palm kernelate | 25% |
| water | to the balance (about 14%) |
| sodium chloride | 0.5% |
| glycerine | 0.5% |
| sodium hydroxide | 0.03% |
| tetra sodium EDTA | 0.02% |
| tetra sodium etidronate | 0.02% |

To this mixture were added 500 g of ESTOL EO3 GC 3606 (PEG 7 glyceryl cocoate, Unichema International) and 250 g PRISORINE GTIS 2041 (Unichema International) stepwise in 5 minutes under continuous mixing. Hereafter, mixing was continued for 5 minutes. The mixture so prepared was extruded in a duplex refiner/plodder to prepare soap tablets.

All resulting tablets had a firm, smooth and homogeneous appearance. In testing, the resulting soap bars appeared to be exceptionally mild to the skin, due to the large amounts of specific moisturizing fluids present in the final bars.

1. Process for the preparation of a soap material comprising the steps of mixing:
a solid phase and
a granular material, characterized in that the granular material comprises at least one fluid phase and a particulate material.

2. Process for the preparation of a soap material comprising the steps of mixing:
a solid phase,
a fluid phase, and characterized in that
a particulate material is incorporated into the mixture.

3. Process according to claim 1 or 2, characterized in that the total amount of fluid is at least 10% by weight, based on the total weight of soap material so produced.

4. Process according to claim 3, characterized in that the total amount of fluid is at least 20% by weight, based on the total weight of soap material so produced.

5. Process according to claim 4, characterized in that the total amount of fluid is at least 25% by weight, based on the total weight of soap material so produced.

6. Process according to claim 5, characterized in that the total amount of fluid is at least 35% by weight, based on the total weight of soap material so produced.

7. Process according to any of claims 1 to 6, characterized in that the total amount of fluid is at most 60% by weight, based on the total weight of soap material so produced.

8. Process according to claim 7, characterized in that the total amount of fluid is at most 50% by weight, based on the total weight of soap material so produced.

9. Process according to claim 8, characterized in that the total amount of fluid is at most 40% by weight, based on the total weight of soap material so produced.

10. Process according to any of claims 1-9, characterized in that the total amount of particulate material in the soap material so produced is at least 2% by weight, based on the total weight of soap material so produced.

11. Process according to any of claims 10, characterized in that the total amount of particulate material in the soap material so produced is at least 10% by weight, based on the total weight of soap material so produced.

12. Process according to any of claims 1-11, characterized in that the particulate carrier material is an anorganic material.

13. Process according to claim 12, characterized in that the particulate carrier material comprises talc, finely ground natural calcium carbonate from limestone, precipitated calcium carbonate, clay, titanium dioxide, silicas/silicates, alumina or combinations of the above.

14. Process according to any of claims 1-13, characterized in that the fluid phase comprises water, glycerol, liquid free fatty acids, liquid fatty acid esters, liquid surfactants, dissolved surfactants, mineral oils, silicon oils, emollients, moisturizers, humectants, perfumes or mixture thereof.

15. Process according to any of claims 1-14, characterized in that the solid phase comprises fatty acid salts and/or synthetic detergents.

16. Process according to any of claims 1-15, characterized in that the soap material is an opaque soap material.

17. Process according to any of claims 1-16 characterized in that the soap material is a soap bar, noodle, pellet, flake or powder.

18. Granules comprising a fluid phase and a particulate material, characterized in that the fluid phase constitutes more than 20% by weight of the granules.

19. Granules according to claim 18, characterized in that it the fluid phase comprises water, glycerol, liquid free fatty acids, liquid fatty acid esters, liquid surfactants, dissolved surfactants, mineral oils, silicon oils, emollients, moisturizers, humectants, perfumes or mixture thereof.

20. Granules according to claim 18 or 19, characterized in that the particulate carrier material is an anorganic material.

21. Granules according to claim 20, characterized in that the particulate carrier material comprises talc, finely ground natural calcium carbonate from limestone, precipitated calcium carbonate, clay, titanium dioxide, silicas/silicates, alumina or combinations of the above.
